(19) United States
(12) Patent Application Publication
(10) Pub. No.: US 2024/0315613 A1
(43) Pub. Date: Sep. 26, 2024

(54) SYSTEM AND METHOD FOR UTILIZING REAL-TIME HEALTH PARAMETER RECORDINGS

(71) Applicant: Know Labs, Inc., Seattle, WA (US)

(72) Inventor: John CRONIN, Williston, VT (US)

(21) Appl. No.: 18/187,390

(22) Filed: Mar. 21, 2023

(51) Int. Cl. A61B 5/145; A61B 5/00; A61B 5/1477

(52) U.S. Cl. CPC A61B 5/14546; A61B 5/1477; A61B 5/4839; A61B 5/7246; A61B 5/742

(57) ABSTRACT

A system which includes an apparatus for generating radio frequency scanning data which includes a transmitter for transmitting radio waves below the skin surface of a person and a two-dimensional array of receive antennas for receiving the radio waves, including a reflected portion of the transmitted radio waves that is reflected from a blood vessel of the person. The transmitter includes at least two different polarization orientations and the receive antennas have polarization orientations that correspond to the transmit antennas. The wave signal is compared to known standard waveforms, and similar waveforms are input into a machine learning algorithm to determine one or more health parameters of the person. The health parameters are recorded and may be later synchronized with a video recording. Alerts may warn health professionals when parameters are outside normal range. The health parameters may be used to direct a device.

### FIELD OF THE DISCLOSURE

The present disclosure is generally related to systems and methods of monitoring health parameters and, more particularly, relates to a system and a method of monitoring a signal that corresponds to the blood glucose level in a user.

### BACKGROUND

Analytes in a patient's blood can change rapidly when undergoing surgery, but currently, not all blood analytes can be accurately detected in real-time without invasive methods.

Variations in blood analytes during a surgical procedure can result in delayed healing, increased wound infection, kidney issues, heart and/or lung problems, neurological complications, stroke, or even death.

Infusion pumps can stabilize the compounds in a patient's blood but may push the concentration too far in the other direction if there is no method of providing feedback.

### DESCRIPTIONS OF THE DRAWINGS

FIG. 1: Illustrates a system for radio frequency health monitoring, according to an embodiment.

FIG. 2: Illustrates an example operation of a Device Base Module, according to an embodiment.

FIG. 3: Illustrates an example operation of an Input Waveform Module, according to an embodiment.

FIG. 4: Illustrates an example operation of a Matching Module, according to an embodiment.

FIG. 5: Illustrates an example operation of a Machine Learning Module, according to an embodiment.

FIG. 6: Illustrates an example operation of a Notification Module, according to an embodiment.

FIG. 7: Illustrates an example of Glucose Waveform, according to an embodiment.

FIG. 8: Illustrates an example of Matching Methods, according to an embodiment.

FIG. 9: Illustrates an example operation of a Sync Playback Module, according to an embodiment.

FIG. 10: Illustrates an example operation of an Infusion Control Module, according to an embodiment.

FIG. 11: Illustrates an example operation of an Alert Module, according to an embodiment.

### DETAILED DESCRIPTION

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

U.S. Pat. Nos. 10,548,503, 11,063,373, 11,058,331, 11,033,208, 11,284,819, 11,284,820, 10,548,503, 11,234,619, 11,031,970, 11,223,383, 11,058,317, 11,193,923, 11,234,618, 11,389,091, U.S. 2021/0259571, U.S. 2022/0077918, U.S. 2022/0071527, U.S. 2022/0074870, U.S. 2022/0151553, are each individually incorporated herein by reference in its entirety.

FIG. 1 illustrates a system for radio frequency health monitoring. This system comprises a body part 102, to which the device 108 is attached or in proximity to. The body part 102 may be an arm 104. The body part 102 may be the other arm of the patient or another body part 106 besides an arm, such as a leg, finger, chest, head, or any other body part from which useful medical parameters can be taken. The system may further comprise the device 108, which may be a wearable and portable device such as, but not limited to, a cell phone, a smartwatch, a tracker, a wearable monitor, a wristband, and a personal blood monitoring device. The device 108 may further comprise a set of TX antennas 110 and RX antennas 170. TX antennas 110 may be configured to transmit the activated RF radio frequency signals at a pre-defined frequency. In one embodiment, the pre-defined frequency may correspond to a range suitable for the human body. For example, the one or more TX antennas 110 transmit activated RF range radio frequency signals at a range of 120-126 GHz. Successively, the one or more RX antennas 170 may be configured to receive the reflected portion of the transmitted activated RF radio frequency signals.

The system may further comprise an ADC converter 112, which may be configured to convert the received activated RF radio frequency signals from an analog signal into a digital processor readable format. The system may further comprise memory 114, which may be configured to store the transmitted activated RF radio frequency signals by the one or more TX antennas 110 and receive a reflected portion of the transmitted activated RF radio frequency signals from the one or more RX antennas 170. Further, the memory 114 may also store the converted digital processor readable format by the ADC converter 112. The memory 114 may include suitable logic, circuitry, and/or interfaces that may be configured to store a machine code and/or a computer program with at least one code section executable by the processor 118. Examples of implementation of the memory 114 may include, but are not limited to, Random Access Memory (RAM), Read Only Memory (ROM), Hard Disk Drive (HDD), and/or a Secure Digital (SD) card.

The system may further comprise a standard waveform database 116, which may contain standard waveforms for known patterns. These may be raw or converted device readings from patients or persons with known conditions. For example, the standard waveform database 116 may include raw or converted device readings from the patient, for example the right arm, known to have diabetes or an average of multiple patients. This data can be compared to readings from a person with an unknown condition to determine if the waveforms from that person match any of the known standard waveforms.

The system may further comprise a processor 118, which may facilitate the operation of the device 108 according to the instructions stored in the memory 114. The processor 118 may include suitable logic, circuitry, interfaces, and/or code that may be configured to execute a set of instructions stored in the memory 114.

The system may further comprise comms 120, which may communicate with a network. Examples of networks may include, but are not limited to, the Internet, a cloud network, a Wireless Fidelity (Wi-Fi) network, a Wireless Local Area Network (WLAN), a Local Area Network (LAN), Long Term Evolution (LTE), and/or a Metropolitan Area Network (MAN).

The system may further comprise a battery 122, which may power hardware modules of the device 108. The device 108 may be configured with a charging port to recharge the battery 122. Charging of the battery 122 may be achieved via wired or wireless means.

The system may further comprise a device base module 124, which may be configured to store instructions for executing the computer program on the converted digital processor readable format of the ADC converter 112. The device base module 124 may be configured to facilitate the operation of the processor 118, the memory 114, the TX antennas 110 and RX antennas 170, and the comms 120. Further, the device base module 124 may be configured to create polling of the activated RF radio frequency signals. It can be noted that the device base module 124 may be configured to filter the activated RF radio frequency signals received from one or more RX antennas 170.

The system may further comprise an input waveform module 126, which may extract a radio frequency waveform from memory. This may be the raw or converted data recording from the RX antennas 170 from a patient wearing the device 108. If the entire radio frequency is too long for effective matching, the input waveform module 126 may select a time interval within the data set. This input waveform may then be sent to the matching module 128.

The system may further comprise a matching module 128, which may match the input waveform and each of the standard waveforms in the standard waveform database 116 by performing a convolution and/or cross-correlation of the input waveform and the standard waveform. These convolutions and/or cross-correlations are then sent to the machine learning module 130.

The system may further comprise a machine learning module 130 which has been trained to identify health parameters based on the convolution and/or cross-correlations of the input and standard waveforms. The machine learning module 130 receives the convolutions and cross-correlations from the matching module 128 and outputs any health parameters identified. The system may further comprise a notification module 132, which may determine if any of the health parameters output by the machine learning module 130 require a notification. If so, the patient and/or the patient's medical care providers may be notified.

In some embodiments, the device base module 124 may utilize a motion module 158 that includes at least one sensor from the group of an accelerometer, a gyroscope, an inertial movement sensor, or other similar sensor. The motion module 158 may have its own processor or utilize the processor 118 to calculate the user's movement. Motion from the user will change the blood volume in a given portion of their body and the blood flow rate in their circulatory system. This may cause noise, artifacts, or other errors in the real-time signals received by the RX antennas 170. The motion module 158 may compare the calculated motion to a motion threshold stored in memory 114. For example, the motion threshold could be movement of more than two centimeters in one second. The motion threshold could be near zero to ensure the user is stationary when measuring to ensure the least noise in the RF signal data. When calculated motion levels exceed the motion threshold, the motion module 158 may flag the RF signals collected at the time stamp corresponding to the motion as potentially inaccurate. In some embodiments, the motion module 158 may compare RF signal data to motion data over time to improve the accuracy of the motion threshold. The motion module 158 may alert the nurse, doctor, or medical staff, such as with an audible beep or warning or a text message or alert to a connected mobile device. The alert would signal the nurse, doctor, or medical staff that the patient is moving too much to get an accurate measurement. The motion module 158 may update the standard waveform database 116 with the calculated motion of the user that corresponds with the received RF signal data. In this manner, the motion module 158 may be simplified to just collect motion data and allow the device base module 124 to determine if the amount of motion calculated exceeds a threshold that would indicate the received RF signal data is too noisy to be relied upon for a blood glucose measurement.

The device base module 124 may utilize a body temperature module 160 that includes at least one sensor from the group of a thermometer, a platinum resistance thermometer (PRT), a thermistor, a thermocouple, or another temperature sensor. The body temperature module 160 may have its own processor or utilize the processor 118 to calculate the temperature of the user or the user's environment. The user's body temperature, the environmental temperature, and the difference between the two will change the blood volume in a given part of their body and the blood flow rate in their circulatory system. Variations in temperature from the normal body temperature or room temperature may cause noise, artifacts, or other errors in the real-time signals received by the RX antennas 170. The body temperature module 160 may compare the measured temperature to a threshold temperature stored in memory 114. For example, the environmental temperature threshold may be set at zero degrees Celsius because low temperatures can cause a temporary narrowing of blood vessels which may increase the user's blood pressure. When the measured temperature exceeds the threshold, the body temperature module 160 may flag the RF signals collected at the time stamp corresponding to the temperature as potentially being inaccurate. In some embodiments, the body temperature module 160 may compare RF signal data to temperature data over time to improve the accuracy of the temperature threshold. The body temperature module 160 may alert the nurse, doctor, or medical staff, such as with an audible beep or warning or a text message or alert to a connected mobile device. The alert would signal to the nurse, doctor, or medical staff that the patient's body temperature, or the environmental temperature is not conducive to getting an accurate measurement. The body temperature module 160 update the standard waveform database 116 with the measured user or environmental temperature that corresponds with the received RF signal data. In this manner, the body temperature module 160 may be simplified to just collect temperature data and allow the device base module 124 to determine if the temperature measure exceeds a threshold that would indicate the received RF signal data is too noisy to be relied upon for a blood glucose measurement.

The device base module 124 may utilize an ECG module 164 that includes at least one electrocardiogram sensor. The ECG module 164 may have its own processor or utilize the processor 118 to record the electrical signals that correspond with the user's heartbeat. The user's heartbeat will impact blood flow. Measuring the ECG data may allow the received RF data to be associated with peak and minimum cardiac output so as to create a pulse waveform allowing for the estimation of blood volume at a given point in the wave of ECG data. Variations in blood volume may cause noise, artifacts, or other errors in the real-time signals received by the RX antennas 170. The ECG module 164 may compare the measured cardiac data to a threshold stored in memory 114. For example, the threshold may be a pulse above 160 bpm, as the increased blood flow volume may cause too much noise in the received RF signal data to accurately measure the blood glucose. When the ECG data exceeds the threshold, the ECG module 164 may flag the RF signals collected at the time stamp corresponding to the ECG data as potentially being inaccurate. In some embodiments, the ECG module 164 may compare RF signal data to ECG data over time to improve the accuracy of the ECG data threshold or to improve the measurement of glucose at a given point in the cycle between peak and minimum cardiac output. The ECG module 164 may alert the nurse, doctor, or medical staff, such as with an audible beep or warning or a text message or alert to a connected mobile device. The alert would signal to the nurse, doctor, or medical staff that the patient's heart rate is not conducive to getting an accurate measurement or requires additional medical intervention. The ECG module 164 may update the standard waveform database 116 with the measured ECG data that corresponds with the received RF signal data. In this manner, the ECG module 164 may be simplified to just collect ECG data and allow the device base module 124 to determine if the ECG data exceeded a threshold that would indicate the received RF signal data is too noisy to be relied upon for a blood glucose measurement.

The device base module 124 may include a received noise module 168 that includes at least one sensor measuring background signals such as RF signals, Wi-Fi, and other electromagnetic signals that could interfere with the signals received by the RX antennas 170. The received noise module 168 may have its own processor or utilize the processor 118 to calculate the level of background noise being received. Background noise may interfere with or cause noise, artifacts, or other errors or inaccuracies in the real-time signals received by the RX antennas 170. The received noise module 168 may compare the level and type of background noise to a threshold stored in memory 114. The threshold may be in terms of field strength (volts per meter and ampere per meter) or power density (watts per square meter). For example, the threshold may be RF radiation greater than 300 μW/m2. When the background noise data exceeds the threshold, the received noise module 168 may flag the RF signals collected at the time stamp corresponding to background noise levels as potentially being inaccurate. In some embodiments, the received noise module 168 may compare RF signal data to background noise over time to improve the accuracy of the noise thresholds. The received radiation module may alert the nurse, doctor, or medical staff, such as with an audible beep or warning, a text message, or an alert to a connected mobile device. The alert would signal to the nurse, doctor, or medical staff that the current level of background noise is not conducive to getting an accurate measurement. The received noise module 168 may update the standard waveform database 116 with the background noise data that corresponds with the received RF signal data. In this manner, the received noise module 168 may be simplified to just collect background noise data and allow the device base module 124 to determine if the measure exceeded a threshold that would indicate the received RF signal data is too noisy to be relied upon for a blood glucose measurement, or if an alternative transfer function should be used to compensate for the noise.

In embodiments, one or more of memory 114, standard waveform database 116, input waveform module 126, matching module 128, the machine learning module 130, the notification module 132, the analyte adjust module 134, the motion module 158, the body temperature module 160, the ECG module 164, and/or the received noise module 168 can be provided on one or more separate devices. In such embodiments, the comms 120 can be used to communicate with other devices to access the memory 114, standard waveform database 116, input waveform module 126, matching module 128, the machine learning module 130, the notification module 132, the analyte adjust module 134, the motion module 158, the body temperature module 160, the ECG module 164, and/or the received noise module 168 by way of any suitable network.

The system may further comprise an admin network 138, which may be a computer or network of computers which collect data from the device 108 and which may have one or more processors for running program instructions and one or more memory storage for storing data. The system may further comprise a device record database 140, which may contain a record of the readings from the device 108 along with a timestamp. Readings may refer to the raw waveform data, health parameter data, or both. If the patient is known, the data in the device record database 140 for that patient may be added to the patient's medical history. For example, readings taken from patient Bob Smith may be added to Bob Smith's medical history. The system may further comprise a video record database 142, which may contain a video recording of a surgical procedure from the camera 152. Surgical procedures can include pre-operative preparation, the performance of the surgical operation itself, and post-operative activities such as suturing, recovery from anesthesia, disinfection of the patient, and the like. The video recording of the surgical procedure can be a video of the surgical procedure as a whole or of portions thereof, such as some or all of the pre-operative preparation, some or all of the operation itself, some or all of the post-operative activities, combinations thereof, and the like. The video may be timestamped so that the readings from the device 108 and the recording from the camera 152 can be synchronized. The system may further comprise a sync playback module 144, which may allow a connected terminal 148 or some other display device to playback the video recording of a surgical procedure while simultaneously displaying the readings from the device 108 during the surgical procedure. For example, the playback may show the video from the camera 152 with the readings from the device 108 overlayed as text or as a graph in the corner of the video. The time may be synchronized such that the health parameter displayed at a given time in the video is the health parameter recorded directly from the patient at that time. Synchronized video and health parameter data may be used for training, including instructional videos, virtual reality, or augmented reality training. Synchronized video and heath parameter data may be used as evidence of malpractice or lack thereof in legal disputes or for insurance purposes. Synchronized video and health parameter data may be used for research purposes.

The system may further comprise an infusion control module 146, which may control a medical device, such as the infusion pump 154, which pumps saline or other chemicals into the patient's bloodstream during surgery. The infusion control module 146 may use the data from the device 108 to adjust the amount or dosage of certain chemicals being infused. For example, if the patient's glucose levels fall below safe levels, the infusion pump may infuse additional glucose into the patient. The system may further comprise a terminal 148, for example a computer with a display. The display may display health parameter data from the device 108 so that a doctor or medical staff can monitor a patient. The computer may have a processor and memory to run an alert module 150. The terminal may or may not be one of the computers that are part of the admin network 138. The terminal 148 may be a user device such as a personal computer, smartphone, or smartwatch. The system may further comprise an alert module 150 that may alert doctors and/or other medical staff when health parameters fall outside normal ranges. The alert may be auditory, visual, haptic, text-based, or any other alert method which may inform doctors and/or other medical staff. The alert module 150 may have a default range for each health parameter which may be customizable by a user. There may be multiple alerts for the same health parameter. For example, there may be a moderate alert for parameters that fall just outside normal levels and an emergency alert for parameters that fall far outside normal.

The system may further comprise a camera 152, which may record surgical procedures. The recording may be stored in the video record database 142. The system may further comprise an infusion pump 154, which may be a medical device that is used to deliver fluids, such as medications, into a patient's body in a controlled manner. During surgical procedures, the infusion pump 154 may be used to administer anesthesia and pain medication, maintain fluid balance and blood pressure, deliver antibiotics or other medications to prevent infection, etc. The infusion pump 154 can be programmed to deliver a specific amount of medication over a certain time and can also be used to deliver fluids continuously.

The system may further comprise a cloud 156 or communication network, which may be a wired and/or wireless network. The communication network, if wireless, may be implemented using communication techniques such as Visible Light Communication (VLC), Worldwide Interoperability for Microwave Access (WiMAX), Long Term Evolution (LTE), Wireless Local Area Network (WLAN), Infrared (IR) communication, Public Switched Telephone Network (PSTN), Radio waves, and other communication techniques known in the art. The communication network may allow ubiquitous access to shared pools of configurable system resources and higher-level services that can be rapidly provisioned with minimal management effort, often over the Internet, and relies on sharing of resources to achieve coherence and economies of scale, like a public utility, while third-party clouds enable organizations to focus on their core businesses instead of expending resources on computer infrastructure and maintenance.

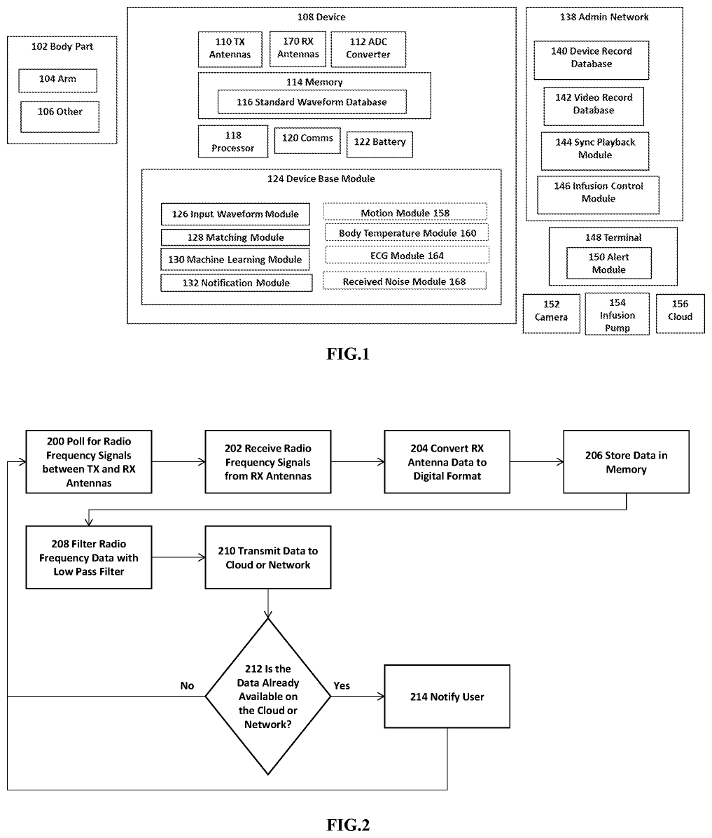

FIG. 2 illustrates an example operation of the device base module 124. The process begins with the device base module 124 polling the activated RF radio frequency signals between the one or more TX antennas 110 and the one or more RX antennas 170, at step 200. The device base module 124 may be configured to read and process instructions stored in the memory 114 using the processor 118. For example, the device base module 124 sends activated RF radio signals of frequency range 120-126 GHz to a TX antenna and stores the activated RF radio signals into the memory 114. The TX antenna sends activated RF range radio signals underneath a patient's skin. The device base module 124 may receive the activated RF radio frequency signals received by the one or more RX antennas 170 at step 202. For example, an RX antenna receives a reflected activated RF radio signal of frequency range 100-110 GHz from the patient's blood. The device base module 124 may be configured to convert the received activated RF radio frequency signals into a digital format using the ADC 112 at step 204. For example, the received activated RF radio signal of frequency range 100-110 GHz is converted into a 10-bit data signal. The device base module 124 may be configured to store converted digital format into the memory 114 at step 206. The device base module 124 may be configured to filter the stored activated RF radio frequency signals at step 208. The device base module 124 may be configured to filter each activated RF radio frequency signal using a low pass filter. For example, the device base module 124 filters the activated RF radio signal of frequency range 100-110 GHz to the activated RF radio signal of frequency range 122-126 GHz. The device base module 124 may be configured to transmit the filtered activated RF radio frequency signals to the cloud or other network using the comms module 120 at step 210. For example, the device base module 124 transmits an activated RF radio signal of frequency range 122-126 GHz to the cloud. The device base module 124 may be configured to determine whether the transmitted data is already available in the cloud or other network at step 212. The device base module 124, using the comms 120, communicates with the cloud network to determine that the transmitted activated RF radio signal of frequency range 122-126 GHz is already available. The device base module 124 may determine that the transmitted data is not already present in the cloud. The device base module 124 may be redirected back to step 200 to poll the activated RF radio frequency signals between the one or more TX antennas 110 and the one or more RX antennas 170. For example, the device base module 124 determines that the transmitted activated RF radio signal of frequency range 122-126 GHz is not present in the cloud, and corresponding to the transmitted signal, there is no data related to the blood glucose level of the patient. The device base module 124 may determine that transmitted data is already present in the cloud. For example, the device base module 124 reads cloud notification of the patient's blood glucose level as 110 mg/dL corresponding to an activated RF radio signal of 122-126 GHz frequency. The device base module 124 may continue to step 214. The device base module 124 may notify the nurse, doctor, or medical staffvia the device 108 of health information, for example, blood glucose level.

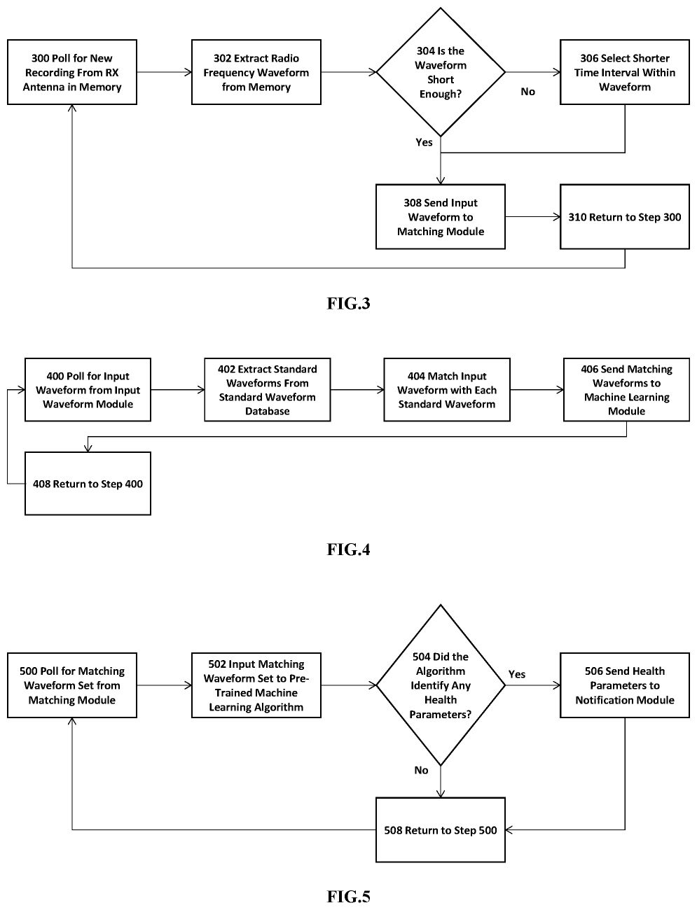

FIG. 3 illustrates an example operation of the input waveform module 126. The process may begin with the input waveform module 126 polling, at step 300, for newly recorded data from the RX antennas 170 stored in memory 114. The input waveform module 126 may extract, at step 302, the recorded radio frequency waveform from memory. If there is more than one waveform recorded, the input waveform module 126 may select each waveform separately and loop through the following steps. The input waveform module 126 may determine, at step 304, if the waveform is small enough to be an input waveform for the matching module 128. This will depend on the computational requirements and/or restrictions of the matching module 128. If the waveform is short enough, the input waveform module 126 may skip to step 308. If the waveform is too long, the input waveform module 126 may select, at step 306, a shorter time interval within the entire recorded waveform. For example, if the waveform is 5 minutes long, then only a 30-second interval may be selected. The interval may be selected at random or by a selection process. The input waveform module 126 may send, at step 308, the input waveform to the matching module 128. The input waveform module 126 may return, at step 310, to step 300.

FIG. 4 illustrates an example operation of the matching module 128. The process may begin with the matching module 128 polling, at step 400, for an input waveform from the input waveform module 126. The matching module 128 may extract, at step 402, each standard waveform from the standard waveform database 116. The matching module 128 may match, at step 404, the input waveform with each standard waveform. Matching may be determining which standard waveforms the input waveform is similar to. Matching may involve convolution and/or cross-correlation of the waveforms or any other suitable matching technique. Cross-correlation and convolution are mathematical operations that can be used to determine the similarity between two wave functions. They are often used in signal processing and image recognition applications to find patterns or features in data. Cross-correlation is a measure of the similarity between two signals as a function of the time lag applied to one of them. It is defined as the integral of the product of two signals after one is flipped and delayed by some amount. By running the cross-correlation function on two wave functions, the output will give a value of similarity between two signals, where the highest value represents the most similar pair. Convolution, on the other hand, is a mathematical operation that combines two functions to produce a third function. It is the integral of the product of two functions after one of them is flipped and then shifted. By applying convolution on two wave functions, the output will be a function in which values represent the degree of similarity between input signals, where higher values represent more similar signals. These operations may be used in combination with other techniques, such as the Fourier transform, to extract information from signals and compare them. Matching waveforms may be waveforms where the cross-correlation and/or convolution values are close to 1 with respect to time. For example, the threshold value may be 0.85. Any point in the function that results from cross-correlation above 0.85 may indicate that the standard waveform matches the input waveform. Matching standard waveforms, the input waveform, the cross-correlation of both, and/or the convolution of both may be used as an input to the machine learning algorithm of the machine learning module 130. The matching module 128 may send, at step 406, the matching waveforms to the machine learning module 130. Matching waveforms may refer to the standard waveforms that were similar to the input waveform, the waveforms that were generated via convolution and/or cross-correlation, or both. The matching module 128 may return, at step 408, to step 400.

FIG. 5 illustrates an example operation of the machine learning module 130. The process may begin with the machine learning module 130 polling, at step 500, for a set of matching waveforms from the matching module 128. Matching waveforms may be a set of standard waveforms that are similar to the input waveform or statistical combinations of the input waveform and standard waveforms, such as convolutions or cross-correlations. The machine learning module 130 may input, at step 502, the set of received waveforms into a pre-trained machine learning algorithm. The machine learning algorithm may be trained on similar sets of matched waveforms where the input waveform is from a patient whose health parameters are known. The waveforms may be input directly into the algorithm, such as a set of X and Y values. The matching waveforms may each be summarized as a closest fit function or may be transformed into a set of sine waves using a Fourier transform. Training data should be labeled with the correct output, such as the type of waveform. In order to prepare the data, the waveforms need to be processed and converted into a format that can be used by the algorithm. Once the data is prepared, the algorithm is trained on the labeled data. The model uses this data to learn the relationships between the waveforms and their corresponding outputs. During training, the model will adjust its parameters to minimize errors between its predictions and the correct outputs. Once the model has been trained and fine-tuned, it can be used to recognize waveforms in new, unseen data. This could be done by giving the input waveforms, then the algorithm will predict the health parameters. The machine learning module 130 may determine, at step 504, if the algorithm identified any health parameters. Identification may require a certain interval of confidence. For example, if the machine learning algorithm determines that it is more than 70% likely that a health parameter is correct, then that parameter may be considered identified. If multiple conflicting parameters exist, then the most confident may be used. For example, if the algorithm determines that it is 75% likely that the patient's blood glucose level is between 110-115 mg/dL and 90% likely that the patient's blood glucose is between 105-110 mg/dL, then the more confident value of 105-110 mg/dL may be identified. If none of the results from the machine learning algorithm are above the confidence threshold, or the results are otherwise inconclusive, the machine learning module 130 may skip to step 508. If any health parameters were identified, the machine learning module 130 may send, at step 506, the health parameters to the notification module 132 and the admin network 138. The machine learning module 130 may return, at step 508, to step 500.

FIG. 6 illustrates an example operation of the notification module 132. The process may begin with the notification module 132 polling, at step 600, for health parameters identified by the machine learning module 130. The notification module 132 may notify, at step 602, the nurse, doctor, or medical staff of the device and/or their care providers. For example, the device may display a readable interface with the identified health parameters such as heart rate, blood pressure, blood glucose, oxygen level, etc. This information may be sent via the comms 120 to another device, such as a terminal in a nursing station, doctor's office, emergency medical transport office, etc. Notification may include audio or haptic feedback such as beeping or vibrating. The notification module 132 may return, at step 604, to step 600.

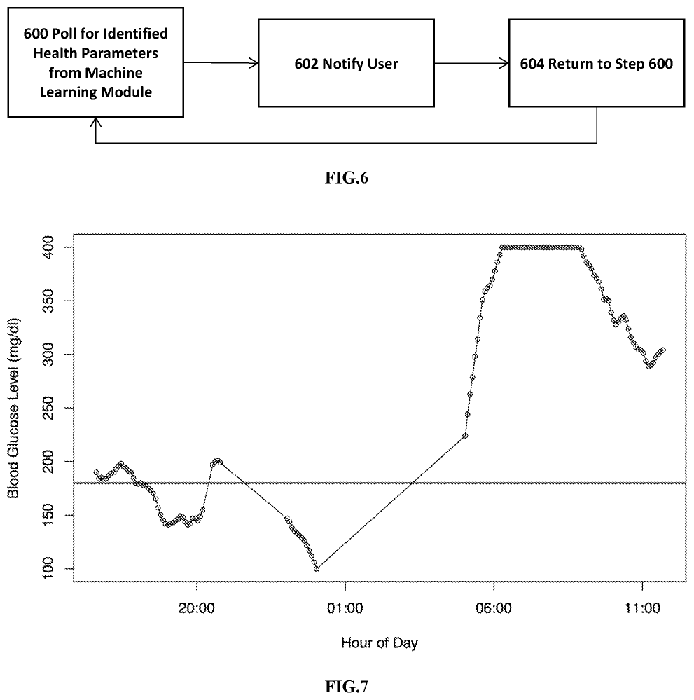

FIG. 7 displays an example of glucose waveform. an example of a glucose waveform. The figure shows blood glucose levels in a patient recorded over time. A computer can store a waveform by digitizing the analog signal and storing the resulting digital values in memory. Digitization is typically accomplished by an analog-to-digital converter (ADC), which samples the amplitude of the analog signal at regular intervals and converts each sample to a digital value. The resulting digital values and information about the sampling rate and bit depth can be used to reconstruct the original waveform when the data is played back. The digital values could be stored in an array or binary files. The computer may store the important parts of the waveform, such as local and/or absolute maxima and minima, inflection points, inversion points, average value, best fit line or function, etc.

FIG. 8 displays an example of matching methods such as convolution and cross-correlation. The figure illustrates two different matching methods, convolution, and cross-correlation. In the convolution process, the standard waveform slides over the input waveform, element-wise multiplying and summing the overlapping values. The result is a new output waveform. The convolution operation is useful for detecting specific features, such as edges, in the input waveform. In the cross-correlation process, the standard waveform is also sliding over the input waveform, element-wise multiplying and summing the overlapping values. However, the output waveform is not generated by summing the product of the standard waveform and the overlapping part of the input waveform but by taking the dot product of the standard waveform and the input waveform. The cross-correlation operation is used to find patterns in the input waveform that are similar to the standard waveform. Convolution and cross-correlation are similar operations used for waveform processing and pattern recognition. They are widely used in image processing, machine learning, computer vision, and waveform processing applications. This is a general description; these methods' actual implementation will depend on the specific use case and application.

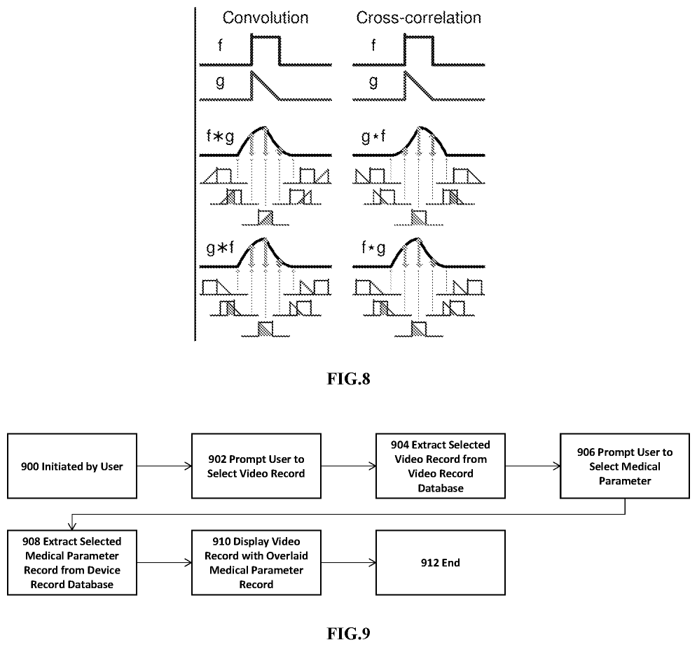

FIG. 9 illustrates an example operation of the sync playback module 144. The process may begin with the sync playback module 144 being initiated, at step 900, by a user. This user may be a doctor, medical staff, medical student, teacher, etc. The user may initiate the module 144 through a computer on the admin network 138 or through a device connected to the admin network, such as a terminal 148. The sync playback module 144 may prompt, at step 902, the user to select a video record of a surgical procedure that was recorded in the video record database 142. The sync playback module 144 may extract, at step 904, the selected video record from the video record database 142. The sync playback module 144 may prompt, at step 906, the user to select a medical parameter such as blood glucose level, SPO2, heart rate, blood pressure, or any other medical parameter which is recorded in the device record database 140. The sync playback module 144 may extract, at step 908, the selected medical parameter with the same timestamp as the selected video record. For example, if the user selected a surgery performed on Jan. 1, 2023 at 8:30 am and the user selected blood glucose level as the medical parameter, then the record of blood glucose level that is also timestamped on Jan. 1, 2023 at 8:30 am may be selected. There may be alternate ways to relate the device records with the video records, such as a naming convention or storing the data in a single data structure such as a database or array. The sync playback module 144 may display, at step 910, the selected video record with an overlay of the selected medical parameter. For example, the display may show the surgery video with the patient's blood glucose levels overlayed as text or as a graph in the corner of the video for instructional purposes. The video may be displayed via a display on one of the computers comprising the admin network 138 and/or on or attached to a terminal 148. The sync playback module 144 may periodically check that the two recordings are synchronized as the combined video is displayed. The sync playback module 144 may end at step 912.

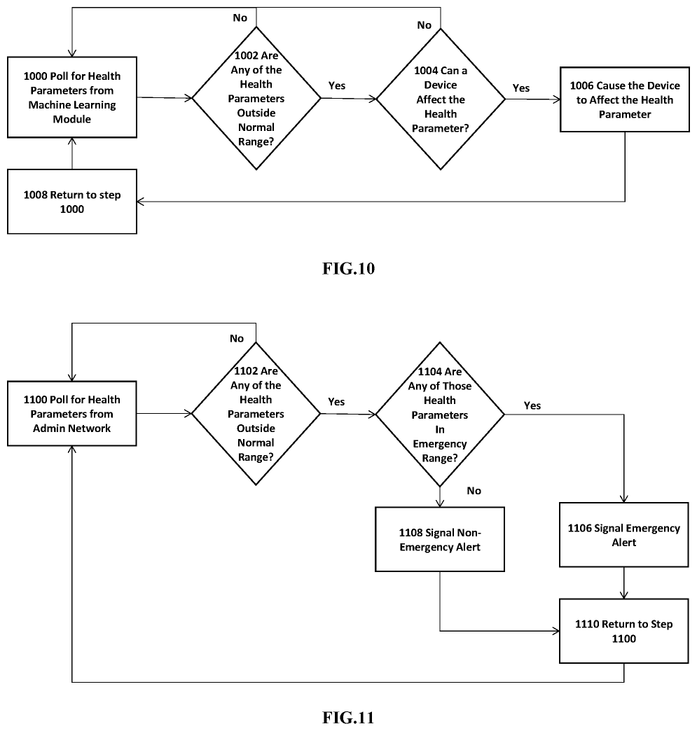

FIG. 10 illustrates an example operation of the infusion control module 146. The process may begin with the infusion control module 146 polling, at step 1000, for health parameters from the machine learning module 130. Health parameters may include heart rate, blood glucose level, blood oxygen level, blood pressure, or any other measurable health metric. The infusion control module 146 may determine, at step 1002, if any of the health parameters are outside the normal range. For example, while fasting, the normal blood glucose range is 80-100 mg/dL. If a patient had a glucose level below 80 mg/dL, that parameter would be outside the normal range. If no parameters are outside the normal range, the infusion control module 146 may return to step 1000. If any health parameters are outside the normal range, the infusion control module 146 may determine, at step 1004, if any available devices can affect the health parameter. For example, if the health parameter is blood pressure, the infusion pump 154 would be able to affect the patient's blood pressure if it could infuse blood pressure medication. If the health parameter is blood oxygen, the infusion pump may not be equipped to infuse oxygen into or out of the patient's blood, but a respirator may be used to increase blood oxygen. If a device is unable to affect the health parameter, the infusion control module 146 may return to step 1000. If a device can affect the health parameter, the infusion control module 146 may adjust, at step 1006, the infusion rate of the chemical or chemicals that may affect the health parameter. For example, if the health parameter outside the normal range is glucose, then the infusion control module 146 may adjust the glucose infusion rate or a chemical that affects glucose level. If the patient's blood glucose is too low, the infusion control module 146 may increase the rate of glucose infused by the infusion pump 154. For another example, if multiple health parameters are outside the normal range, such as the electrolytes (sodium, potassium, chlorine, etc.), then the infusion control module 146 may adjust the infusion rate of saline or another electrolyte mixture. The infusion control module 146 may increase, decrease, stop, start, or otherwise adjust the infusion of a chemical. The adjustment may be reverted once the health parameters reach normal levels. The infusion control module 146 may return, at step 1008, to step 1000.

FIG. 11 illustrates an example operation of the alert module 150. The process may begin with the alert module 150 polling, at step 1100, for health parameters from the admin network 138 or directly from the machine learning module 130. Health parameters may include heart rate, blood glucose level, blood oxygen level, blood pressure, or any other measurable health metric. The alert module 150 may determine, at step 1102, if any of the health parameters are outside the normal range. For example, while fasting, the normal blood glucose range is 80-100 mg/dL. If a patient had a glucose level below 80 mg/dL, that parameter would be outside the normal range. The normal range may be a default or manually entered by a user. For example, for a known diabetic patient, a doctor could set the normal range of blood glucose to 90-110 mg/dL because that is normal for that patient. For another example, a surgical procedure may be more sensitive to slight changes in glucose level, so a doctor may set the normal range to 85-95 mg/dL. If no parameters are outside the normal range, the alert module 150 may return to step 1100. If any parameters are outside the normal range, the alert module 150 may determine, at step 1104, if any, the health parameters are in the emergency range. For example, blood glucose below 55 mg/dL calls for immediate action. There may be multiple emergency ranges. If the health parameter is in emergency range, the alert module 150 may signal, at step 1106, an emergency alert. The alert may include auditory, visual, haptic, text-based, or any other alert method which may inform doctors and/or other medical staff. This alert may be more noticeable than a non-emergency alert. For example, if blood glucose drops below 40 mg/dL, a shrill alarm may signal any nearby medical staff. If the health parameter is not in the emergency range but is still outside the normal range, the alert module 150 may signal, at step 1108, a non-emergency alert. The alert may include auditory, visual, haptic, text-based, or any other alert method which may inform doctors and/or other medical staff. The alert module 150 may return, at step 1110, to step 1100.

The functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

1. A method of health monitoring of a patient during a surgical procedure, the method, comprising:
non-invasively determining a real-time level of an analyte in a body of the patient during the surgical procedure, wherein non-invasively determining the real-time level of the analyte in the body of the patient includes:
transmitting, using one or more transmit antennas, a transmit signal into the body of the patient during the surgical procedure;
receiving a response signal responsive to the transmit signal, wherein the response signal is a radio frequency signal;
processing the response signal to determine the real-time level of the analyte in the body of the patient;
recording a video of the surgical procedure during the surgical procedure on the patient;
generating a synchronized video including the video of the surgical procedure and a display of a real-time parameter at a corresponding time of the video of the surgical procedure, wherein the real-time parameter is based on the real-time level of the analyte in the body of the patient; and
providing an alert during the surgical procedure based on the real-time level of the analyte in the body of the patient, wherein the alert is determined by determining whether the real-time parameter is within a range, the range based on a sensitivity of the body of the patient during the surgical procedure to the real-time level of the analyte in the body of the patient.

2. The method of claim 1, wherein the real-time parameter comprises the real-time level of the analyte.

3. The method of claim 1, wherein the real-time parameter is a health parameter determined based on the real-time level of the analyte.

4. The method of claim 3, wherein determining the health parameter includes matching the response signal to one or more standard waveforms of a standard waveform database and applying a machine learning algorithm to the matched response signal and the one or more standard waveforms.

5. The method of claim 1, wherein the synchronized video is an augmented reality or virtual reality presentation.

6. (canceled)

7. The method of claim 1, wherein the alert is provided based on an extent of deviation from a predetermined level of the real-time parameter.

8. The method of claim 1, wherein the alert includes one of an auditory alert, a visual alert, or a haptic alert.

9. The method of claim 1, further comprising displaying the synchronized video.

10. The method of claim 1, further comprising controlling an infusion pump based on the real-time parameter, wherein the infusion pump is configured to provide a chemical to the patient.

11. A surgical care system, comprising:
a non-invasive radio frequency analyte monitoring device that is configured to non-invasively obtain real-time readings of a level of a first analyte in a body of a patient during a surgical procedure using radio frequency signals; and
a processor, configured to:
receive the real-time readings of the level of the first analyte in the body of the patient during the surgical procedure from the non-invasive radio frequency analyte monitoring device;
receive a video recording of the surgical procedure; and
generate a synchronized video including the video recording of the surgical procedure and a display of a real-time parameter at a corresponding time of the video of the surgical procedure, wherein the real-time parameter is based on the real-time readings of the level of the first analyte in the body of the patient during the surgical procedure,
wherein the processor is configured to determine an alert during the surgical procedure based on the real-time readings of the level of the first analyte in the body of the patient, the alert is determined by determining whether the real-time parameter is within a range, the range based on a sensitivity of the body of the patient during the surgical procedure to the level of the first analyte in the body of the patient.

12. The surgical care system of claim 11, further comprising a display configured to display the synchronized video.

13. The surgical care system of claim 11, wherein the real-time parameter comprises the real-time readings of the level of the first analyte in the body of the patient.

14. The surgical care system of claim 11, wherein the real-time parameter is a health parameter determined based on the real-time readings of the level of the first analyte in the body of the patient.

15. The surgical care system of claim 14, further comprising a standard waveform database, and wherein the processor is configured to determine the health parameter by matching a response signal to one or more standard waveforms of the standard waveform database and applying a machine learning algorithm to the matched response signal and the one or more standard waveforms.

16. (canceled)

17. The surgical care system of claim 11, further comprising a camera, the camera configured to obtain the video recording of the surgical procedure.

18. The surgical care system of claim 11, further comprising an infusion pump connected to and controlled by the processor.

19. The surgical care system of claim 18, wherein the processor is configured to control the infusion pump based on the real-time parameter.
